(19) United States
(12) Patent Application Publication
(10) Pub. No.: US 2009/0148928 A1
(43) Pub. Date: Jun. 11, 2009

(54) Heterotrophic Shift

(76) Inventors: Cheryl A. Hackworth, San Jose, CA (US); Thomas W. Chalberg, JR., Redwood City, CA (US)

Correspondence Address: WILSON SONSINI GOODRICH & ROSATI, 650 PAGE MILL ROAD, PALO ALTO, CA, 94304-1050, US

(21) Appl. No.: 12/324,583

(22) Filed: Nov. 26, 2008

Related U.S. Application Data
(60) Provisional application No. 60/991,201, filed on Nov. 29, 2007.

(51) Int. Cl. C12N 1/12

(52) U.S. Cl. 435/257.3; 435/257.1

(57) ABSTRACT

Methods and systems of cultivating photosynthetic cells under autotrophic and heterotrophic growth conditions are described herein. Under different growing conditions, photosynthetic cells may produce different quantities and characteristics of lipids. The methods and systems herein utilize changing growth conditions to alter the macromolecular content of a photosynthetic cell.

### CROSS-REFERENCE

This application claims the benefit of U.S. Provisional Application No. 60/991,201, filed Nov. 29, 2007, which application is incorporated herein by reference in its entirety.

### BACKGROUND OF THE INVENTION

Mass cultivation of algae has been used for decades for water treatment and for creating nutritional supplements, fertilizer, and food additives. In recent years, commercial growth of algae has also been explored to create biologically-derived energy products such as biodiesel, bioethanol, and hydrogen gas. When compared to terrestrial crops that can be used for biofuels, such as corn, soybeans, and sugarcane, algae can grow much faster and can produce up to 30 times more biomass per acre than the next most efficient crop. Unlike terrestrial plants, which have roots and leaves, algae biomass is generally less specialized, and most or all cells can be used in conversion to fuel. The macromolecular makeup of the cellular matter can be an important determinant of the quantity and quality of products obtained from photosynthetic organisms.

For the production of oils or biofuel, for example, it can be desirable to harvest organic compounds from organisms that contain a greater amount of lipids as a proportion of the total biomass. It can also be desirable to alter the macromolecular composition of cellular matter to a more optimal lipid profile, for example to produce oil with greater energy density or lower viscosity, which in turn can produce higher quality fuel.

Previous work has used nutrient starvation (such as N or Si limited growth) to induce a change in the lipid composition of plant cells such as microalgae (“A Look Back at the US Dept of Energy's Aquatic Species Program: Biodiesel from Algae.” NREL, 1998). Though this process successfully alters the macromolecular composition of cells, it does not generally result in greater productivity since the resulting algae culture grows more slowly in the nutrient-limited condition.

Inducing a more favorable macromolecular makeup and cellular composition without greatly sacrificing overall productivity of cell growth represents an advance in the art.

### SUMMARY OF THE INVENTION

In an aspect, the invention provides a method for altering the macromolecular content of a photosynthetic cell comprising utilizing a shift from autotrophic to heterotrophic or mixotrophic growth conditions, thereby altering said macromolecular content of said photosynthetic cell.

In another aspect of the invention, a method is provided for altering the quantity of lipids in a photosynthetic cell comprising utilizing a shift from autotrophic to heterotrophic or mixotrophic growth conditions, thereby altering the quantity of lipids in said photosynthetic cell. In an embodiment, the quantity of lipids in said photosynthetic cell is increased.

In an aspect, a method for altering the character of lipids in a photosynthetic cell comprises utilizing a shift from autotrophic to heterotrophic or mixotrophic growth conditions, thereby altering the character of lipids in a photosynthetic cell. The altered character of lipids can be a more desirable fuel or fuel precursor than a character of lipids from a photosynthetic cell grown in autotrophic growth conditions.

A photosynthetic cell can be an algal cell. In an embodiment, the algal cell is a green algal cell. In a further embodiment, the green algal cell is a cell from a species of Chlorella.

In an aspect of the invention, a method is provided for maturing algal cells comprising moving algal cells from a first growth condition to a second growth condition, wherein said first growth condition comprises a growth medium with no source of organic carbon, and wherein said second growth condition comprises growth medium containing a source of organic carbon.

In an embodiment, the moving algal cells further comprises: a) removing said algal cells from the first condition; and b) transferring said algal cells to the second condition.

In another embodiment, the second condition is similar to said first condition with the addition of a source of organic carbon.

In an embodiment, a method of maturing a photosynthetic cell can further comprise maturing the lipids of said algal cell.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

A better understanding of the features and advantages of the invention will be obtained by reference to the following detailed description that sets forth illustrative embodiments, in which the principles of the invention are utilized, and the accompanying drawings of which:

FIG. 1 demonstrates methods and systems sequentially utilizing both autotrophic growth and heterotrophic growth to obtain the advantages of both growth processes.

FIG. 2 demonstrates an exemplary system of the invention wherein algae are grown in a plurality of modular PBRs under autotrophic conditions and can be transferred to a single larger chamber that provides heterotrophic growth conditions for the organisms.

FIG. 3 illustrates a defined amount of heterotrophic medium that is used for heterotrophic growth, resulting in 15 arbitrary units of useful energy.

### DETAILED DESCRIPTION OF THE INVENTION

While embodiments of the invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

Methods and systems are described herein for altering the macromolecular composition of cells by shifting the culture medium from autotrophic to heterotrophic or mixotrophic conditions. The methods can be useful with any photosynthetic organism that can grow in at least two of autotrophic, heterotrophic, and mixotrophic conditions. In an embodiment, the photosynthetic organism is an algal species.

In an aspect, a method and system disclosed herein can increase the proportion of lipids in a photosynthetic cell. In an embodiment, the methods and systems further comprise improving the character of those lipids to make them more optimal for uses of biomass oils including fuel.

An autotroph can be defined as an organism that produces complex organic compounds from simple inorganic molecules and an external source of energy, such as light or chemical reactions of inorganic compounds. Photosynthetic organisms take energy from sunlight and are often referred to as phototrophs (or photoautotrophs). Autotrophic growth of a photosynthetic organism can be defined as biological growth that uses only sunlight as an energy source to convert inorganic carbon (such as CO₂) to organic compounds (such as hydrocarbons). Typically, to grow a photoautotrophic organism, such as an algal species, the growth mechanism requires salts in a medium (such as nitrates, phosphates, and small amounts of metals) and carbon dioxide or a dissolved inorganic carbon as a carbon source.

A heterotroph can be defined as an organism that requires organic substrates as a carbon source for growth and development. Heterotrophic growth can be defined as biological growth that uses organic molecules as an energy source. These organic molecules can be derived from plant or animal cells or can take the form of a sugar or starch. In an embodiment, algal heterotrophic growth uses glucose as an energy source. In some embodiments, a heterotrophic growth medium can be similar to autotrophic growth medium with an addition of about 5% glucose. In many cases, cells grown in heterotrophic conditions are grown without light. Because the heterotrophic cells are using sugar as the energy source, the carbon products that result from the breakdown of sugar are typically used as the primary carbon source in contrast to autotrophic cells which use carbon dioxide as a primary carbon source.

A mixotroph can be described as an organism (usually algae or bacteria) capable of deriving metabolic energy both from photosynthesis and from external energy sources, often simultaneously. These organisms may use light as an energy source, or may take up organic or inorganic compounds. They may take up simple compounds osmotically (by osmotrophy) or by engulfing particles (by phagocytosis or myzocytosis). Mixotrophic growth can involve providing both a light energy source and an organic carbon source for biological growth of an organism.

The invention pertains to organisms that can grow under at least two of autotrophic, heterotrophic, or mixotrophic conditions. In an embodiment, the organism is a green algal species. Other examples of species that can grow under at least two of autotrophic, heterotrophic, or mixotrophic conditions include, but are not limited to, algae (for example, green and red algae), vascular plants (for example, tobacco, Arabidopsis, ferns), and prokaryotic cyanobacteria. In an embodiment, the cells or organisms have been genetically modified. For example, the organism can be an organism that has been genetically modified to be photosynthetic, an organism that has been modified to grow mixotrophically or heterotrophically, or an organism that has been modified to create or alter a substance that is not naturally produced by that organism.

A photosynthetic organism or biomass, as used herein, includes all organisms capable of photosynthetic growth, such as plant cells and microorganisms in unicellular or multi-cellular form that are capable of growth in a liquid phase. These terms may also include organisms modified by natural selection, selective breeding, directed evolution, synthetic assembly, or genetic manipulation. While applications disclosed herein are particularly suited for the cultivation of algae, one skilled in the art can recognize that other photosynthetic organisms may be utilized in place of or in addition to algae.

Typically, when growing a large amount of an organism that can grow under at least two of autotrophic, heterotrophic, or mixotrophic conditions, growing the organism under purely autotrophic conditions can be the most energy efficient method and most cost-effective method of growth since all of the energy is derived from the sun. Under autotrophic conditions, however, most plants generally contain only a modest proportion of lipids as a percentage of total cell mass. Heterotrophic growth, by contrast, offers a different lipid profile that impacts both the quantity and character of the lipids produced in a cell of the organism. In some settings, these advantages may be enough to offset the added cost of the supplied carbon source necessary for heterotrophic growth. Lipid quantities and character are especially important in the production of biofuels.

Chlorella is an example of a photosynthetic species that contains a different lipid profile when grown under autotrophic as compared to heterotrophic conditions. This example is intended as illustrative and is not necessarily limited to Chlorella or algae, or even any one genus or type of algae, as would be understood by those with ordinary skill in the art. For example, Chlorella cells grown in autotrophic conditions contain ˜14% lipids of the total cell mass, while heterotrophically grown cells contained ˜55% lipids of the total cell mass, an increase of approximately four-fold in heterotrophic growth conditions (“High yield bio-oil production from fast pyrolysis by metabolic controlling of Chlorella protothecoides,” Miao and Wu, Journal of Biotechnology, 2004, 110: 85-93).

The physical characteristics and relative quantity of the lipids, deemed herein as the lipid profile, are also known to differ under autotrophic and heterotrophic growth. Lipids from heterotrophically-grown cells more closely approximate that of petroleum-based diesel fuels than do lipids from autotrophically grown cells in a number of ways. A few non-limiting examples are listed in Table 1.

TABLE 1

| | Autotrophically- | | Heterotrophically- | | Petroleum-derived | |
|---|---|---|---|---|---|---|
| Property | Grown Chlorella | | Grown Chlorella | | diesel fuel | |
| Density | 1.06 kg per liter | | 0.92 kg per liter | | 0.75-1.0 kg per liter | |
| Viscosity | 0.10 | Pa-s | 0.02 | Pa-s | 2-1000 | Pa-s |
| Heating | 30 | MJ/kg | 41 | MJ/kg | 42 | MJ/kg |
| value | | | | | | |

| | | | |
|---|---|---|---|
| Oxygen | Higher | Lower | Lower |
| content | | | |

Based on the quantity and character of lipids produced, prior art has proposed that the best method to culture algae for the production of biofuels is heterotrophic growth.

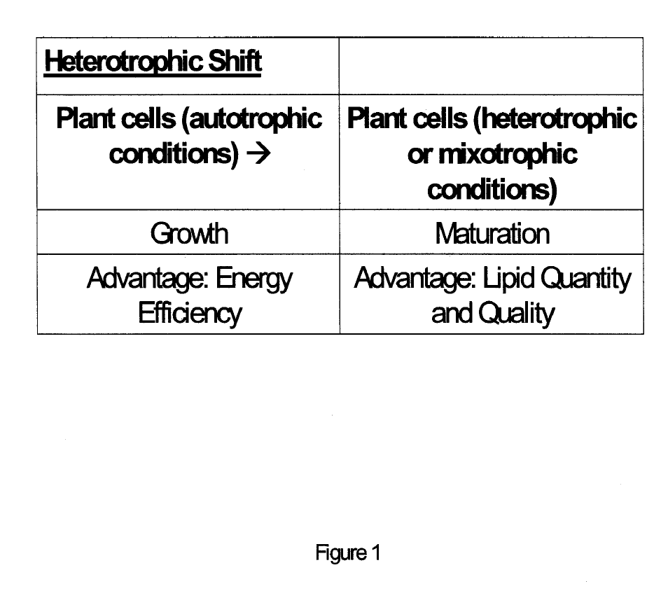

The methods and systems herein utilize the advantages of both autotrophic growth (capturing the sun's energy and drawing carbon dioxide out of the atmosphere) and heterotrophic growth (producing a more desirable quantity and character of lipids). Combining the two techniques does not simply mean growing the cells mixotrophically (in sunlight with sugar). Typically, photosynthetic organisms such as algae, when given the option in mixotrophic growth, choose to use the sugar as a carbon and energy source. The invention provides methods and systems sequentially utilizing both autotrophic growth and heterotrophic growth to obtain the advantages of both growth processes as shown in FIG. 1.

In an aspect of the invention, a method comprises growing photosynthetic cells in autotrophic conditions to capture the sun's energy and atmospheric carbon dioxide. The cells would then undergo a “lipid maturation phase” in which a source of organic carbon is added. This second step can be performed without any available sunlight (heterotrophic conditions), or in the presence of sunlight (mixotrophic conditions).

Cells can be first grown to dense logarithmic phase under autotrophic conditions in a clear growing chamber, and can then pumped to a dark chamber with no available sunlight. Sugar (or other organic sugar or carbohydrate molecule, such as corn or rice sugar powder, or carbohydrates derived from algal biomass) can then be pumped into the chamber at a concentration of about 5%, inducing heterotrophic growth of the cells. When a method invention is practiced, the heterotrophic growth, after only a limited number of cell division, can cause a change in lipid composition. In an embodiment, the end result is a dense culture that has derived most of its energy from the sun, has obtained most of its carbon from the atmosphere, and contains lipids that are best suited for biodiesel production and use.

By continuously growing algae in autotrophic conditions, all of the generated cellular energy is derived from inorganic carbon, making the process very energy and financially efficient. However, the resulting lipid content of the cells may be lower in total percentage and lower in specific desired lipid forms. Growth in heterotrophic medium can increase total percentage of lipid produced and alter the ratio of lipids to favor those desired forms. However, growth in heterotrophic medium requires an input of sugar, adding to the cost of production of these lipid products. In a method of the invention, the above two growth conditions can be combined in series, with growth first in autotrophic conditions to optimize input efficiency, and then shifted briefly to heterotrophic conditions just prior to lipid extraction to optimize total lipid yield and desired lipid content.

Additionally, a method of the invention may have desirable effects on the composition of other macromolecules. For example, a cell may produce more complex sugar molecules which may be useful as commercial products. This is within the scope of the invention and can be considered a practice of the invention.

In an aspect of the invention, a system is provided that for growing a photosynthetic organism in the presence of light and then changing the growth conditions to provide an organic carbon source to the organism. In an embodiment, a photobioreactor (PBR) system can be utilized to change growth conditions for an organism. The photosynthetic organism can be grown in any suitable growing system including, but not limited to, open ponds, covered ponds, photobioreactors, bioreactors, Petri dishes, Erlenmeyer flasks or other similar vessels, and the ocean.

In an embodiment, a photosynthetic organism can be grown under autotrophic conditions utilizing either an external or internal light source to the growing system. After a certain period of time, an organic carbon source can be added, thus beginning heterotrophic growth and the lipid maturation phase. In an embodiment, when an organic carbon source is added to a PBR, light energy is still provided to the organism, which creates mixotrophic growth conditions. Alternatively, light energy can be eliminated from the system, creating heterotrophic growth conditions.

In an alternative embodiment, a photosynthetic organism is grown under autotrophic conditions for a certain period of time in a system, and then the organism is transferred to a second system that provides an organic carbon source to the organism. The organism can grow heterotrophically in the second system and begin the lipid maturation phase. In an embodiment, light energy is still provided to the organism, thereby creating mixotrophic growth conditions. Alternatively, light energy can be eliminated from the system, creating heterotrophic growth conditions.

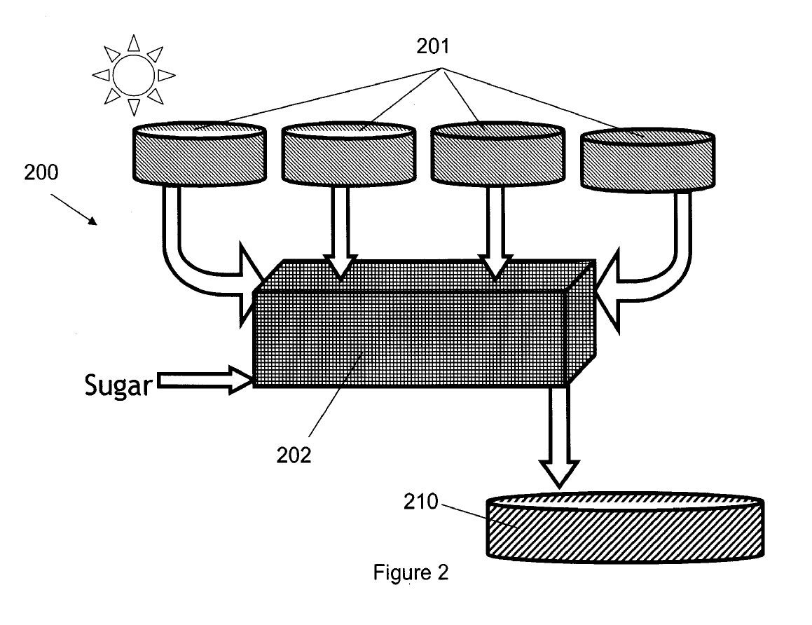

In another embodiment, photosynthetic organisms are grown in a plurality of ponds, chambers, or PBRs under autotrophic conditions, and after a certain time, the organisms are then transferred to a second bioreactor that provides heterotrophic or mixotrophic growth conditions. FIG. 2 demonstrates an exemplary system 200 of the invention. In the example, algae are grown in a plurality of modular PBRs 201 under autotrophic conditions. Autotrophically grown algae can be transferred to a single larger chamber 202 that provides heterotrophic growth conditions for the organisms. The transfer of the algae can be performed in series, semi-continuous, or continuous mode to the lipid maturation chamber 202. After another period of time, the algae can be harvested from the lipid maturation chamber 202 and the lipids 210 can be collected and utilized for various processes including the production of biodiesel or other commercially useful products.

A plurality of autotrophic chambers, such as ponds or photobioreactors, can be arranged to form a system for the growth and production of a photosynthetic biomass. As would be apparent to those skilled in the art, in some embodiments, a photobioreactor system can comprise one of a plurality of identical or similar photobioreactors interconnected in parallel, in series, or in a combination of parallel and series configurations. For example, this could increase the capacity of the system (e.g., for a parallel configuration of multiple photobioreactors). The plurality of autotrophic chambers can also be coupled to a plurality of lipid maturation chambers or a single lipid maturation chamber that provide heterotrophic or mixotrophic growth conditions for improving the lipid content and/or characteristics of the biomass. In an embodiment, instead of transferring the biomass to a second bioreactor, an organic carbon source is added to the plurality of PBRs to create mixotrophic growth conditions. The PBRs can also be covered and provided with no light energy to create heterotrophic growth conditions for the photosynthetic biomass. All such configurations and arrangements of the inventive photobioreactor apparatus provided herein are within the scope of the invention.

Each unit of a system of the invention can operate independently. The units can be modular and they can be easily swapped if desired. For example, if one unit becomes contaminated with another species of algae or other organism, it can be swapped for a different unit.

Although a system of the invention can be intended to be modular and self-contained, harvest processes, medium recycling, water storage, power generation, and other processes may be centralized and distributed to individual units. Independent units can be connected in a network so that dispersal of medium and collection of biomass products can be centrally coordinated.

In some embodiments a control system and methodology is utilized in the operation of a system, which is configured to enable automatic, real-time optimization and/or adjustment of operating and growth parameters to achieve a shift from autotrophic to heterotrophic (or mixotrophic) growth conditions. In yet another aspect, the invention involves methods and systems for preselecting, adapting, and conditioning one or more species of photosynthetic organisms to specific environmental and/or operating conditions to which the photosynthetic organisms will subsequently be exposed during utilization of a system of the invention.

### EXAMPLE 1

One of the aspects of the invention involves generating the “desired products” (useful energy, or E_{useful}) following a shift from autotrophic to heterotrophic growth in a greater quantity than the desired products resulting from purely heterotrophic (HT) or autotrophic (AT) growth.

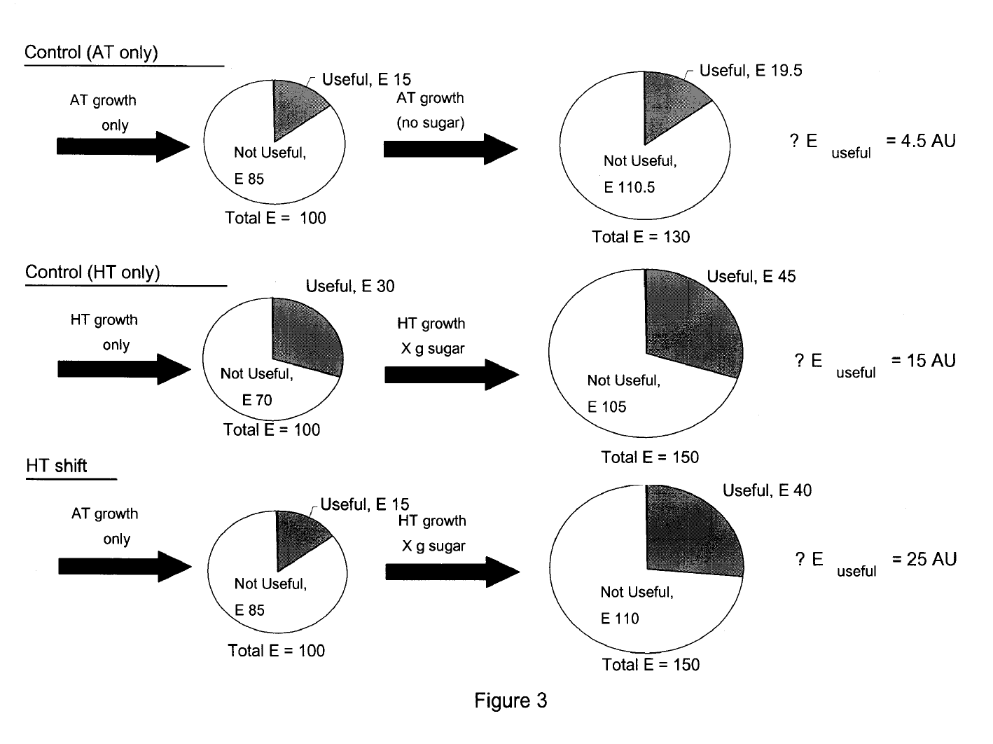

Therefore a successful practice of the invention would yield ΔE_{useful}(AT→HT)>ΔE_{useful}(HT→HT), as in the example in FIG. 3. In FIG. 3, a defined amount of heterotrophic medium (X g sugar) is used for HT growth, resulting in 15 arbitrary units (AU) of E_{useful}. This results in more total growth over a comparable period than the AT only case, and a greater proportion of useful products. With HT shift, however, X g of sugar fuels growth to 150 AU, as well as a shift in the macromolecular makeup of the cell, from 15% E_{useful} to 27% E_{useful}. The result expected is that 25 AU of E_{useful} are created in the case utilizing a method of the invention shifting from AT to HT growth. Thus the amount of sugar used by a method of the invention case creates a greater amount of E_{useful} than the HT only case.

Such a case occurs, and a method invention can be practiced, when heterotrophic growth medium drives not only the synthesis of new useful products, but either or both of: a) disproportionate synthesis of useful products compared to HT growth alone, and b) the conversion of not useful products to useful products. One could hypothesize that in the heterotrophic growth environment, resources are abundant, which drives the cell toward storage of energy-rich products in case they are needed later. At the same time, under heterotrophic growth, the cell does not need to continue to produce photosynthetic proteins that, are no longer required, also anticipating a shift away from proteins and toward energy-dense storage products.

### EXAMPLE 2

How is E_{useful} defined/measured in an experimental setting? The benefits of heterotrophic shift can be tested experimentally as described above. Importantly one needs to define the quantity E_{useful} and develop an assay to measure it.

In one embodiment, E_{useful} can be defined as the total amount of lipid in the culture. This could be assayed in a number of ways including:

E_{useful}=(# cells)*(% lipid per cell)

where % lipid is assayed by the number and size of lipid vesicles viewed under a microscope, or

E_{useful}=(# cells)*(% lipid per cell)

where % lipid is assayed by staining (e.g., using NILE red) and quantified by visualization under a microscope or using a spectrophotometer to measure staining

In an alternative embodiment, E_{useful} can be defined as a subset of lipid, for example, those most useful for fuel, such as saturated fatty acids, in the culture. This could be assayed in a number of ways including:

E_{useful}=(mg of plant matter)*(amt of unsaturated fatty acids per mg)

where the amount of saturated fatty acids is quantified by mass spectrometry, or

E_{useful}=(mg of plant matter)*(amt of unsaturated fatty acids per mg)

where the amount of saturated fatty acids is quantified by silicic acid columns via differential elution followed by Si gel thin layer chromatography according to the method of Tornabene (Tomabene et al, 1982 as referenced in NREL p. 29).

A subset of lipid can be defined as useful by testing it in a practical application, such as verifying lipid content is optimized for biodiesel use in mechanical engines by obtaining biodiesel certification for the lipid product.

What is claimed is:

1. A method for altering the macromolecular content of a photosynthetic cell comprising utilizing a shift from autotrophic to heterotrophic or mixotrophic growth conditions, thereby altering said macromolecular content of said photosynthetic cell.

2. A method for altering the quantity of lipids in a photosynthetic cell comprising utilizing a shift from autotrophic to heterotrophic or mixotrophic growth conditions, thereby altering the quantity of lipids in said photosynthetic cell.

3. The method of claim 2, wherein the quantity of lipids in said photosynthetic cell is increased.

4. A method for altering the character of lipids in a photosynthetic cell comprising utilizing a shift from autotrophic to heterotrophic or mixotrophic growth conditions, thereby altering the character of lipids in a photosynthetic cell.

5. The method of claim 4, wherein said altered character of lipids is a more desirable fuel or fuel precursor than a character of lipids from a photosynthetic cell grown in autotrophic growth conditions.

6. The method of claim 1, 2, or 4, wherein said photosynthetic cell is an algal cell.

7. The method of claim 6, wherein said algal cell is a green algal cell.

8. The method of claim 6, wherein said algal cell is cell from a species of Chlorella.

9. A method for maturing algal cells comprising moving algal cells from a first growth condition to a second growth condition, wherein said first growth condition comprises a growth medium with no source of organic carbon, and wherein said second growth condition comprises growth medium containing a source of organic carbon.

10. The method of claim 9, wherein said moving algal cells further comprises: a) removing said algal cells from the first condition; and b) transferring said algal cells to the second condition.

11. The method of claim 9, wherein said second condition is similar to said first condition with the addition of a source of organic carbon.

12. The method of claim 9 further comprising maturing the lipids of said algal cell.
